(19) United States
(12) Patent Application Publication
(10) Pub. No.: US 2014/0303509 A1
(43) Pub. Date: Oct. 9, 2014

(54) METHOD AND APPARATUS FOR NON-INVASIVE DETERMINATION OF CARDIAC OUTPUT

(71) Applicant: Duncan Campbell Investments Pty Ltd, Beecroft (AU)

(72) Inventor: Duncan Islay Campbell, Beecroft (AU)

(21) Appl. No.: 14/310,229

(22) Filed: Jun. 20, 2014

Related U.S. Application Data
(63) Continuation of application No. PCT/AU2012/000854, filed on Jul. 17, 2012.

(30) Foreign Application Priority Data
Jan. 30, 2012 (AU) 2012900322

(51) Int. Cl. A61B 5/021; A61B 5/00; A61B 5/029; A61B 5/024

(52) U.S. Cl. CPC A61B 5/02108; A61B 5/02444; A61B 5/6826; A61B 5/029
USPC 600/485

(57) ABSTRACT

A non-invasive method and apparatus determines continuously cardiac output by first analysing the trace obtained from an optical sensor which has been scaled and calibrated using an electronic sphygmomanometer. From this the mean arterial pressure and time constant are determined. Compliance is determined from the pulse delay between two other optical sensors at well separated sites. Cardiac output is the product of mean arterial pressure and compliance divided by the time constant. A microcomputer provides the necessary calculations.

This application is a continuation of International application No. PCT/AU2012/000854 filed on Jul. 17, 2012 and claims the benefit of Australian application No. 2012900322 filed on Jan. 30, 2012, which are incorporated herein by reference in their entirety and for all purposes.

### BACKGROUND OF THE INVENTION

The invention is described in the following statement:

1. Technical Field

Cardiac output is the volumetric rate at which blood is expelled from the left ventricle of the heart. The present invention relates to a non-invasive method of, and apparatus for, determining cardiac output. Knowledge of cardiac output is important for the diagnosis and treatment of many medical conditions and is of particular value in the operating theatre where continuous monitoring allows early detection and treatment of cardiovascular problems.

2. Background Art

At present an accepted method of measuring cardiac output derives the cardiac output from a thermo dilution technique using a pulmonary artery catheter. (Swan Ganz, Edwards Laboratories USA. www.edwards.com). This method utilises a catheter advanced via the great veins through the right heart chambers and pulmonary valve into the pulmonary artery. The procedure is potentially hazardous and is rarely used outside cardio-thoracic units and intensive care areas.

Other invasive methods involve arterial catheterisation with analysis of pressure changes. (LiDCO system,www.LiDCO.com). This company also has an invasive system using a lithium indicator dilution technique. The FloTrac sensor with Vigileo monitor of Edwards Laboratories is also based on analysis of the arterial pressure wave and also uses an invasive intra arterial catheter. A less invasive method is the oesophageal ultrasonic Doppler method, (CardioQ, Deltex Medical Ltd Chichester, UK. www.deltexmedical.com). This ultrasonic Doppler technique obtains cardiac output from an accurately positioned probe in the oesophagus to give flow information from the descending aorta from which cardiac output is calculated.

Non-invasive externally applied methods include magnetic resonance imaging which involves expensive bulky equipment and transcutaneous aortic ultrasonic Doppler (USCOM www.uscom.com.au). These both depend on the use of algorithms based on height and weight of the patient.

Another non-invasive system for deriving cardiac output uses a non-invasive pressure measurement obtained from a finger combined with an algorithm. This is described in patent AU200071581 B2, and uses the Penaz system (U.S. Pat. No. 4,869,261) which is a less accurate pressure measuring system than an invasive arterial measurement and also carries the risk of possible tissue damage from any sustained pressure around the finger; also, errors occur from non-scaling of the derived pressure trace and errors from using an algorithm for deriving compliance.

None of these prior art devices provides a satisfactory solution to the provision of a method or an apparatus for continuous accurate non-invasive measurement of cardiac output. There is a special requirement for such continuous display non invasive systems in operating theatres, especially those theatres where arterial and central venous catheters are not routinely used but major surgery on elderly high risk patients is performed daily. The minimally invasive oesophageal Doppler is sometimes used, but has the disadvantage that it depends on an algorithm, and positioning of the probe is critical with slight movement upsetting accuracy.

First Publication of Cardiac Output Equation

In 1899 a publication by Otto Frank showed that total peripheral arterial resistance may be calculated from the time constant of diastolic aortic pressure decay divided by arterial compliance¹. He stated that cardiac output may then be calculated by dividing mean arterial pressure by the total peripheral arterial resistance. No technology existed at that time to utilise this equation. The present invention provides a non invasive method of utilizing this equation.

### SUMMARY OF THE INVENTION

The present invention seeks to overcome the disadvantages in the prior art and to provide an easier, simpler, accurate and non invasive method and apparatus for obtaining cardiac output from the analysis of pulse wave traces derived by optical or ultrasonic Doppler means. The method determines systemic vascular resistance and this divided into the mean arterial pressure gives cardiac output. Blood pressure is measured intermittently, but not necessarily exclusively, by a non invasive electronic sphygmomanometer² (e.g. Omron Monitor www.omronhealthcare.com.au) so that the systolic and diastolic pressures are used for scaling and calibration of the pulse wave trace which then replicates the cyclical intra arterial pressure changes. From this trace the mean arterial pressure is derived and by using a programmed computer the time constant of the arterial system is derived. From the delay time of the pulse wave, measured by two additional sensors, the compliance of the arterial system is derived. Dividing the time constant by compliance gives the value for the resistance of the arterial system and the mean arterial pressure divided by resistance gives cardiac output. Pulse rate is measured continuously.

Other factors can be determined from these measurements and include stroke volume which is cardiac output divided by heart rate. Cardiac contractility can be derived from the upward slope of the calibrated waveform to display rate of change of pressure, or with a known stroke volume, rate of change of volume. The ejection period may also be derived from the time interval between the start of the upswing of the trace to the dicrotic notch which marks the closure of the aortic valve. Other parameters can be determined from the above measurements and a continuous display of cardiac output and related cardiovascular factors is provided.

### BRIEF DESCRIPTION OF THE DRAWINGS

The present invention will now be described with respect to the following figures in which:

FIG. 1 shows a trace obtained from an optical transducer attached to a finger. The trace has been scaled and calibrated from the systolic and diastolic measurements obtained from a non invasive sphygmomanometer.

FIG. 2 shows simultaneous traces obtained from two optical transducers with one attached to the ear and the other attached to a toe.

FIG. 3 shows a trace obtained from an optical transducer attached to a finger. The trace has been scaled and calibrated from the systolic and diastolic measurements obtained from a non invasive sphygmomanometer.

FIG. 4 shows a block diagram of an apparatus for performing the method according to the invention; and

FIG. 5 shows a flow chart for the apparatus of FIG. 4 for providing the values that can be determined according to the invention.

### DETAILED DESCRIPTION

Sensors Used and an Explanation of the Information Provided.

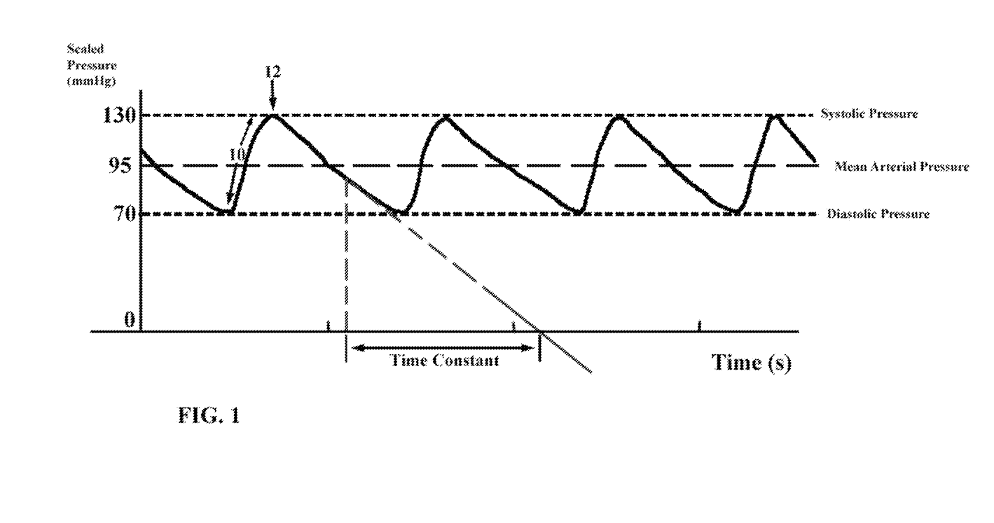

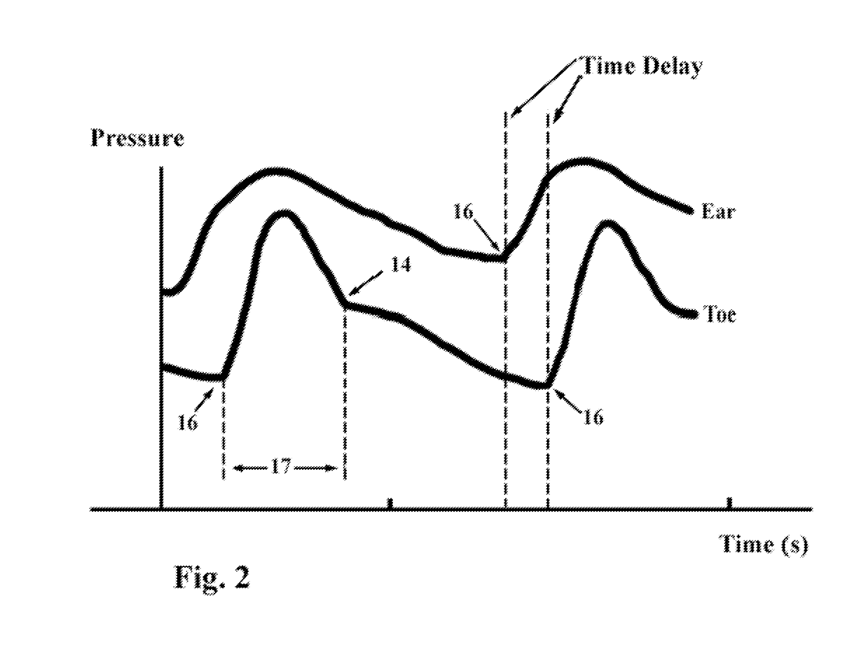

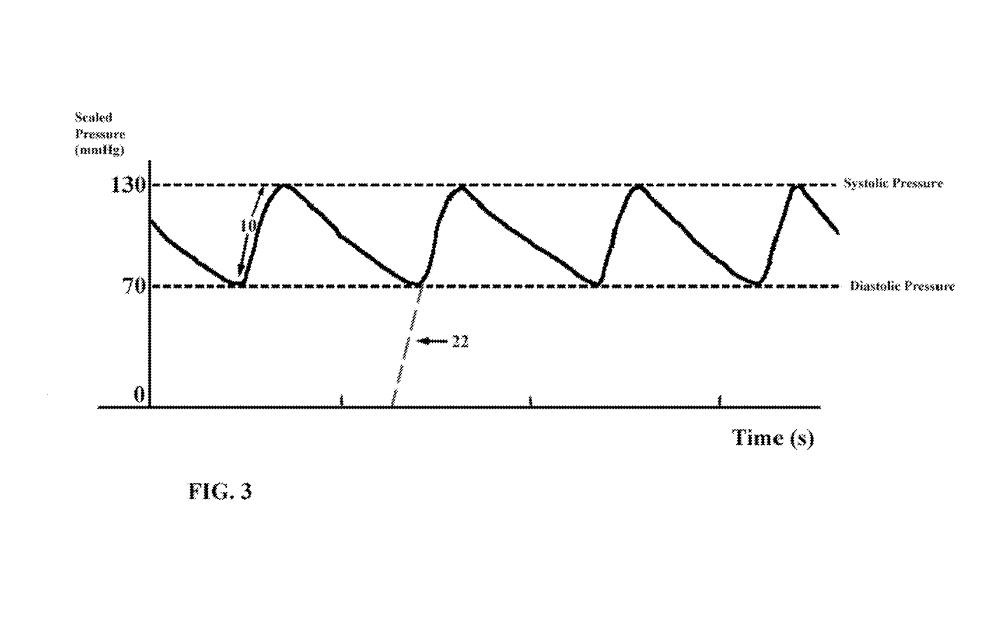

As shown in FIG. 1, the trace obtained from the optical transducer attached to the finger, rises and falls with the activity of the heart and corresponds to an arterial pressure wave obtained from an intra-arterial catheter. The rising portion 10 corresponds to the cardiac ejection phase. The highest point corresponds to the systolic pressure 12. Towards the end of the ejection phase the trace falls. About one third of the time along the descending portion an irregularity called the dicrotic notch often appears, (FIG. 2 14), and this corresponds to closure of the aortic valve with attendant pressure disturbance. The period from the commencement of ventricular contraction until the closure of the aortic valve is the systolic phase (FIG. 2 17), and the period from the closure of the aortic valve to the next ventricular contraction is the diastolic phase. The lowest point of the trace corresponds to the diastolic pressure (FIG. 2 16). FIG. 1 shows the mean arterial pressure and a graphical indication of the measurement of the time constant. FIG. 2 shows the measurement of pulse wave delay which is taken from the beginning of the upswing of each pulse wave. From this delay measurement, compliance is calculated. FIG. 3 shows a measurement of the maximum rate of change of pressure, from which cardiac contractility is calculated. The traces shown in FIGS. 1, 2, and 3 are obtained from optical sensors, each sensor consisting of an infrared light source, with the light passing through the tissues to a photocell. The photocell registers the absorption of the infrared light, this having an absorption characteristic appropriate for blood. These sensors are easily attached to fingers, toes or ears and have been commercially available for many years as for example the infrared heart rate monitor from Kyto Electronic Company Limited, (www.kytocn.com) which detects changes in blood flow and as flow is proportional to pressure the waveform is proportional to the arterial pressure waveform. Frequent calibration and scaling is required to ensure accuracy and this is done with reference to the blood pressure measurements obtained at intervals from an electronic non invasive sphygmomanometer. The optical waveform is free from the reverberations and distortions inherent in a pressure transducer system³. The optical system with a rapid sampling rate gives a clear trace at the beginning of each waveform and this allows accurate timing of pulse wave delay. Freedom from distortions allows accurate measurement of the time constant. Optical sensors do not require hand holding over an arterial vessel as with tonometer type probes (e.g. SphygmoCor www.atcormedical.com). The positioning of optical sensors on digits and the ear is simple and the sensors may be left in position for prolonged periods without need of personnel to hold them in place. Continuous monitoring is therefore possible as for example during surgery, where Doppler probes cannot be used. Optical timing of pulse wave delay may be taken between the ear/finger and ear/toe. The ear to toe measurement involves the passage of the pulse wave along most of the aorta, and as the aorta is the major vessel of the body, pulse delay measurements along this vessel may be preferred.

Some types of optical sensor function with reflected light as opposed to transmitted light, the advantage being that they can be placed on the skin at sites other than fingers, toes and ears. At present the disadvantage is that a more powerful light intensity is used, so that with poor tissue perfusion the heat generated may not be dissipated and burns could result. If this problem is overcome, these would be ideal sensors for restless patients as sensors could be strapped on to regions such as the forehead.

A further non-invasive method for measuring the pulse waves is by using ultrasonic Doppler sensors (Bestman BV-520T with 8 MHz probe for example www.szbestman.com/en). The positioning of these is more critical than the optical sensors and may require holding in position by hand, making them suitable for short term use only. Doppler sensors may provide greater accuracy in timing pulse wave delay as the pulse in the ascending aorta can be timed against for example the pulse of the dorsalis pedis artery in the foot, this as an alternative to the optical ear/toe sensors. Doppler and optical sensors may be combined so that for instance a Doppler sensor registers the pulse in the ascending aorta while an optical sensor is used on the toe. The ascending aorta is immediately above the aortic valve so as this is the first region to receive the pulse wave, time delay measurement to the foot will be longer than ear/foot, and so is potentially more accurate.

Time delay may also be measured using an electrocardiogram [ECG] so that the time delay is measured between the R or S wave of the ECG and the ascending aorta Doppler pulse. After this the ECG can be used to replace the ascending aorta Doppler for timing pulse wave velocity. So for example, measuring the time delay between the ECG R wave and a foot sensor can be converted to ascending aorta to foot delay time by subtracting the previously measured ECG to ascending aorta Doppler time. (The ECG complex is approximately 40 milliseconds before the opening of the aortic valve, this being the isometric period of cardiac activity when pressure inside the ventricle rises with cardiac muscle contraction with the aortic valve remaining closed until the ventricular pressure exceeds the aortic pressure). The advantage of using the ECG is that a continuous signal is delivered without patient discomfort, no extra personnel involved for holding probes, and, possibly a likely mandatory requirement for an ECG during the particular procedure.

A further method for measuring pulse wave delay is the use of a microphone on the chest wall to record the heart sounds. The first heart sound corresponds to the beginning of the ejection period, [and the pulse wave] so can be used for timing purposes combined with a remote optical or Doppler sensor.

Terminology:

Compliance

Compliance of the arterial system is a measure of elasticity measured in volume change per unit pressure change⁴. It is relatively linear over the normal blood pressure range of a healthy individual so that the same measurement would result from a reading taken at 90 mmHg and at 120 mmHg, but the particular value might vary according to conditions such as body temperature and with various medications. For a healthy young adult the compliance of the arterial system is in the region of 1.6 ml/mmHg.

Pulse wave delay is proportional to arterial compliance⁵ so that by measuring the pulse delay along an arterial vessel, compliance can be determined. As the pulse pathway chosen is a proportional section of the total arterial system, the measured pulse delay is proportional to the total systemic arterial compliance. This effectively applies to all adults so that the product of the pulse delay in seconds and a numerical constant gives the value for compliance.

Pulse wave delay measured between the ear and toe is a suitable standard for optical probes, giving a pulse delay which can be timed with greater accuracy than over any shorter distance, and a pulse wave path which passes along the aorta. The aorta is the largest and most compliant part of the arterial system, responsible for much of the total arterial compliance. It has been estimated from the multiple ear to toe pulse delay times measured, that the value of the constant is approximately 13. A healthy 70 Kg young adult male will have a pulse delay over this distance of about 125 milliseconds, so for this timing, the compliance works out as 1.625 ml/mm. Hg.

Greater accuracy of the compliance constant will be achieved with simultaneous measurements of cardiac output measured by this method, and comparing this with measurements using existing accepted invasive methods. (e.g. Swan Ganz, Edwards Laboratories USA). Statistical analysis will then provide a means of improving the accuracy of the said numerical constant used for compliance determination.

Other time delay pathways such as ear to finger may be used when convenience with less accuracy is acceptable, but will require a separate constant, estimated in this case as approximately 18, with this value adjusted empirically after multiple testing.

Pulse delay measurements are made from the beginning of the upswing of each pulse. Inaccuracies occur if peak to peak are taken as there is variable flattening of the crests.

Arterial vascular compliance is an important determinant of cardiac output but has received little attention in most systems for measuring cardiac output and including this value in an algorithm has often been the preferred option. Tonometric measurements are stated as a method for deriving compliance in Patent AU200071581 B2, with hand held probes measuring the pulse delay along an artery from the movement of the vessel wall. The accuracy of this method is unacceptable. This patent application uses optical or Doppler sensors, which register changes in blood flow and have good accuracy. Pulse wave velocity studies as a background to the present invention indicate that there may be considerable changes in compliance from hour to hour, so that during cardiac output monitoring, periodical checks on compliance are essential to avoid errors.

Total Peripheral Vascular Resistance [TPR]

The systemic vascular resistance comprises a number of vascular beds connected in parallel between the arterial and venous systems. The total peripheral resistance is therefore less than the resistance presented by any single organ and is given by the equation: TPR=mean arterial pressure divided by cardiac output. It is usually in the units in which the other two are measured, so that TPR is expressed as mmHg per litre per minute. For example if the cardiac output is 5 litres per minute, and the mean arterial pressure is 100 mmHg, the total systemic vascular resistance would be 20 mmHg per litre per minute. (By using a multiplication factor of 79.9 this value is converted into the less convenient dynes units). From the above equation it is seen that the cardiac output can be determined if the mean arterial pressure and TPR are both known. TPR may be determined in a closed system from the equation TPR=Time Constant/Compliance. The equation for deriving Cardiac output is therefore: Mean arterial pressure×Compliance/Time Constant.

Time Constant and Exponential Functions

With an exponential function the quantity under consideration decreases at a rate proportional to the amount still present. In the peripheral arterial system, after the aortic valve closes, the system becomes a capacitance-resistance combination. The blood pressure falls exponentially and therefore has a time constant which may be measured from the arterial pressure trace. Every exponential process has a time constant. The time constant is the length of time that 100 percent change would take if the initial rate of change were maintained. One time constant is 63%.

In this patent application the time constant is derived from the calibrated trace obtained from the optical sensor as shown in FIG. 1. The lower part of the falling trace is used for this measurement. When the aortic valve closes, the arterial vessels with their volume and elasticity provide the capacitance of the system while the flow through the arterioles is the leakage through a resistance equivalent. The arterial system therefore has an exponential fall in pressure after the aortic valve closes and the time constant of this exponential can be measured using the lower part of the curve so as to ensure that the readings are made after closure of the aortic valve. A visual measurement of the time constant is shown in FIG. 1, with a line drawn as a tangent to the curve and extended to the base level, and a vertical line dropped from the point of intersection of the tangent with the curve to the base line. The time constant is the time interval between the points at which these two lines intersect the base line. The pulse wave is scaled and calibrated from a non invasive electronic sphygmomanometer, or other source, using the systolic and diastolic points so that the base line and mean arterial pressure can both be established. A programmed computer is able to derive the time constant from the slope of the exponential curve or from the time taken to drop a given percentage. Computer analysis of each pulse averaged over several pulses provides greater accuracy than a single measurement, so that for continuous cardiac output monitoring, information can be upgraded on a visual display at for example fifteen second intervals. The pulse wave used is from a region where the tissues are not compressed and there is good perfusion. An optical sensor over a finger nail bed is ideal. The vascular Doppler may also be used for short term use, but for continual monitoring it could be difficult to maintain an accurate position over a vessel.

Exemplary Method of Performing the Invention

One optical sensor is attached to the patient's finger or thumb of either hand, one optical sensor is attached to an ear lobe on either side and one optical sensor is attached to any convenient toe. A non invasive blood pressure system (NIBP equipment such as Omron) is connected to the patient with the cuff preferably applied to the upper arm on the body side which does not have the finger optical sensor. These units are all connected through a control module and computer so that all the required parameters are displayed on a screen. The sensor from the finger provides the trace which is scaled and calibrated from the NIBP systolic and diastolic pressure. The timing interval between blood pressure measurements is set by the operator. The finger trace provides the trace for measuring the time constant. The ear and toe sensors only provide the pulse delay measurements and so do not require scaling. The compliance value derived from these two sensors may remain stable with no requirement for frequent upgrading. The frequency of compliance upgrading may be set by the operator. If less accuracy of compliance is acceptable, and it is more convenient, the toe sensor may be eliminated and the ear to finger pathway used for pulse delay measurements to derive compliance. It is preferable to use the finger trace for scaling and time constant measurements as some distortion may occur with the clip pressure on the ear lobe reducing tissue flow at the diastolic pressure levels, whereas in the finger the bony structure prevents this from occurring. The optical trace from the toe is of inferior quality to the finger trace. With both the ear and the toe, pulse timing remains unaffected by the above problems. It may occasionally be desirable to wrap sensors in disposable sheaths to avoid cross infection. This is particularly important if using the sensors in regions exposed to body secretions such as lips, tongue or nose. In these regions disposable sensors would be preferable, but such occasions would be rare as good signals are normally available from the ear and the digits.

As discussed previously other sensors may be used and may be desirable, so that for instance in the operating theatre, if an ECG is in use, the signal from this might be preferred over the ear lobe sensor for pulse wave timing. Although the invention is non-invasive, on occasions when an arterial line is already in place, it might be preferable to have the flexibility of using the systolic and diastolic output from this source rather than duplicating measurements by applying a NIBP apparatus to the patient. Ultrasonic Doppler probes and microphones may also be used as discussed previously.

For continuous monitoring under stable conditions checking compliance every hour would be satisfactory, but reduced to quarter hourly or every minute in unstable situations. Similarly, electronic blood pressure readings may be taken at half hourly intervals but reduced to five minutes or even every minute if cardiac output is unstable. With continuous monitoring the time constant and pulse rate are continually monitored with averaging for about fifteen seconds before computing the various parameters and updating the information. Cardiac output corrections will be necessary for absent limbs or during surgical procedures when tourniquets are applied or major vessels clamped, as these conditions alter total arterial compliance. The operator must also be aware that if the aortic valve is incompetent, the percentage reflux will reduce the effective cardiac output by that amount. An Ultrasonic Doppler probe can be used to diagnose the existence and degree of incompetence.

Final Calculations

Using these determinants of mean arterial pressure, compliance, and the time constant, cardiac output is derived using the equation previously described by first calculating the total peripheral resistance since the product of compliance and total peripheral resistance equals the time constant determined for the system. The calculated total peripheral resistance can then be used with the measured mean arterial pressure to calculate the mean cardiac output. Having determined the mean cardiac output and pulse rate, the stroke volume is the mean cardiac output divided by the pulse rate. This and further calculations are rapidly effected and displayed on a visual display screen attached to a programmed computer, as the analogue signals from the sensors are converted to digital format. The PICO system (picotech.com) is an example of a method for programming a computer, displaying the traces as with an oscilloscope, performing mathematical functions and displaying results in digital format.

An indication of cardiac contractility is derived from the rate of rise of the waveform (FIG. 3 22). It can then be displayed as rate of change of pressure, or if stroke volume has been calculated, as rate of change of volume. The cardiac index can be derived as this relates the cardiac output to the body surface area based on an existing formula relating height and weight of a patient. With an electrocardiogram connected, the cardiac pre ejection period can be measured and displayed. This is the period between the start of ventricular activity seen with the electrocardiogram, and the commencement of the pulse wave in the ascending aorta. An Ultrasonic Doppler probe on the neck or chest wall will provide the timing of the beginning of the aortic pulse.

Example of Cardiac Output Measurement

A patient has the following derived parameters:

Mean arterial pressure [MAP] 90 mmHg. (This is obtained from the scaled waveform).

Systemic Arterial Compliance [C] 1.6 ml/mmHg. (Obtained from the pulse wave delay and specific multiplier)

Time Constant of Arterial System [t] 1.4 seconds. (Obtained from the exponential pressure drop of a calibrated waveform)

$$\begin{matrix}Cardiac Output = MAP \times C / t ml \text{/} second \\ = 90 \times [ 1.6 / 1.4 ] ml \text{/} second \\ = 90 \times [ 1.6 / 1.4 ] \times [ 60 / 1000 ] Litres \text{/} minute . \\ = 6.17 Litres \text{/} minute .\end{matrix}$$

Usefulness of the Invention

The main role of the invention will be to display moment to moment changes in cardiac output and peripheral resistance, and so will be of great value to anaesthetists for all major surgical procedures especially as the system is easy to use, uses compact equipment which does not interfere with other theatre activities, requires no additional personnel and supplies a continual display of cardiac output as well as other cardiovascular information. As there is difficulty at present in justifying the use of many of the invasive cardiac output monitors in most operating theatres, this new non-invasive invention would be readily accepted and welcomed. Also in view of the ever increasing volume of major surgery on poor risk patients, such monitoring allows early correction of cardiovascular problems, this being important for patient welfare. All changes associated with blood loss or fluid replacement will be seen as will the changes associated with the administration of anaesthetic agents or other drugs. Achieving adequate peripheral perfusion will be greatly facilitated and play an important role in patient management in the operating theatre, intensive care and accident and emergency units. By using this safe non-invasive invention in place of existing invasive systems there will be considerable cost saving by eliminating the need for expensive disposable items as well as time saved in setting up the apparatus. Patient comfort is improved and potential complications associated with invasive systems are eliminated. The use will also extend to use in general wards and will find a place in general practice to check on patient's health and assessing response to treatment. In such cases only a few minutes of recording would be required and the results could be stored and kept for comparison at future visits. The apparatus for determining these factors can be made portable making it useful for on-site emergency procedures as well as increasing the general use of the invention which will also have applications in veterinary practice.

Electronic Controls and Programming

Any microcontroller or microcomputer well known to the technician in the field of electronics or medical electronics can be used as a programmable device to measure and calculate the above stated parameters. The hardware and software components are well known in the art and it is within the skill of a person of average ability in the art to construct a device to determine these parameters or write software to operate the microcontroller or microprocessor to perform these functions.

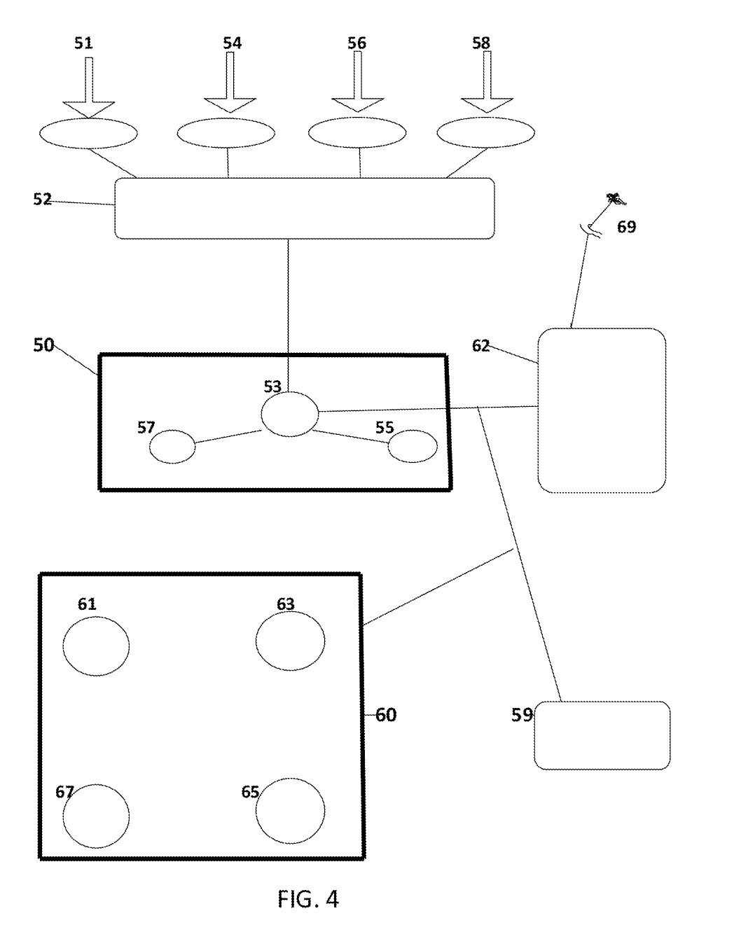

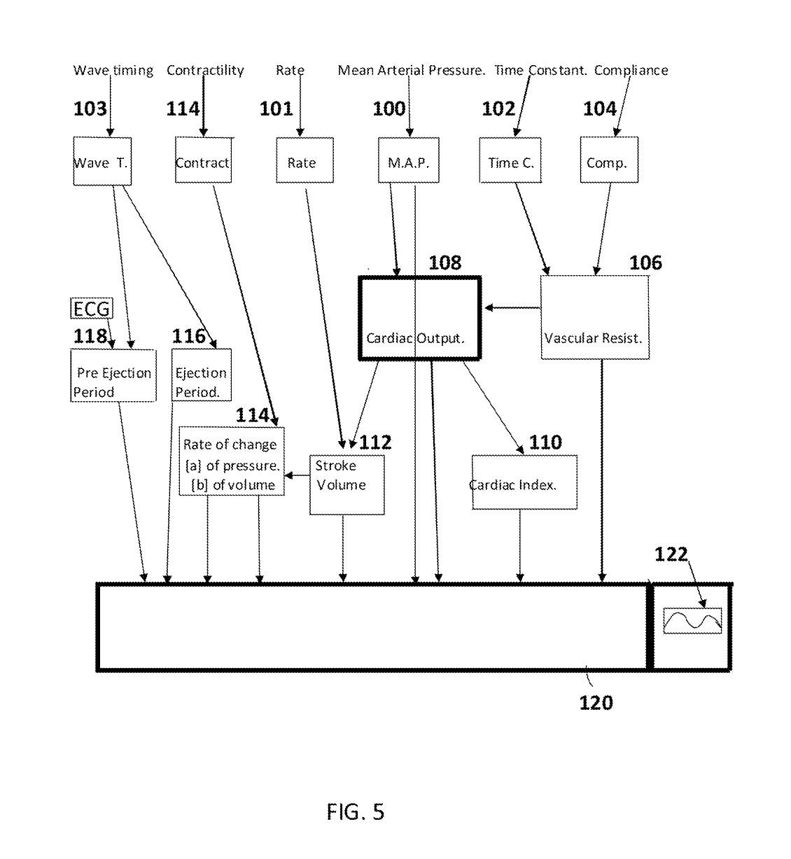

For example as shown in FIG. 4 a typical apparatus involves a computer 50, having an input 52 from sensors 51, 54, 56, 58, corresponding respectively to an electronic sphygmomanometer or other arterial pressure measuring device, one or more optical sensors, one or more Doppler probes and one or more electrocardiographic leads. The computer [50] involves a processing unit [53] operated under software control in RAM 55 or ROM 57 which performs a series of software steps such as set forth in FIG. 5. The output of these software steps provides values which can be displayed as tables 61, 63, 65, 67, in an output device [60] such as a visual display unit, a meter or similar device, a printer [59] or may be input into a further communication device [62] for transmission to a remote location either over a phone line or wireless link [69] including radio, optical fibre, microwave link or the like. A meter may be an electromechanical device or may be a light emitting diode display or liquid crystal display.

Referring to FIG. 5, the series of software steps perform the following operations. The pulse wave trace is input through the input device as discussed above. The trace is converted from an analogue form to a digital form for processing by the computer. Once the trace has been converted to a digital form it is scaled, calibrated and sampled over a period of typically 15 seconds to provide the mean arterial pressure (step 100) The time constant is calculated by analysing the slope of the pulse wave below the dicrotic notch and extrapolating a tangent on the curve to zero to obtain the time constant (step 102). Several such measurements can be taken on each curve and averaged for example over 15 pulse waves. A programmed computer may also derive the time constant by timing the fall from a given point to a percentage drop from that point. (With an exponential fall, one time constant gives a fall to 36.8% of the initial value). The computer may further have available a table of compliances to use for the best match for the trace for example stored in ROM device [57].

The time constant is divided by the compliance [104] to determine the total peripheral resistance [step 106]. The total peripheral resistance can then be used with the mean arterial pressure to determine the cardiac output [step 108]. The cardiac output can then be used to determine a variety of subsequent values including the cardiac index [step 110] and the stroke volume [step 112]

From the optically derived pulse wave, the maximum slope of the rising wave reflects cardiac contractility [114] which can be expressed as rate of change of pressure or rate of change of volume.

Measuring the period from the start of the upswing of the pulse trace to the dicrotic notch provides the ejection period [step 116]. The pre ejection period is measured in conjunction with an electrocardiogram [step 118] and preferably using a Doppler signal from the ascending aorta for precise timing.

The outputs from the calculations or steps can then be displayed in a tabular or numerical or histogram form [120] for interpretation, while the pulse waveform is displayed as an analogue trace [122]. The display of output values[120] may for example include the values of systolic pressure, diastolic pressure, heart rate, mean arterial pressure, compliance, systemic vascular resistance, cardiac output, cardiac index, stroke volume, maximum contractility [expressed as volume and/or pressure change], ejection period and systolic pre ejection period.

Alternatively, these values may be transmitted to a remote location through a telecommunications link [69] for evaluation by a specialist, for example in a road side emergency or a home care environment.

It is not considered necessary to include the exact sequence of steps for calculating the various values in the flow diagram shown with respect to FIG. 5 as this is within the skill of an average programmer or workman in the field.

Although the invention has been herein shown and described in what is conceived to be the most practical and preferred embodiments, it is recognized that departures can be made within the scope of the invention, which are not to be limited to the details described herein but are to be accorded the full scope of the appended claims so as to embrace any of the equivalent assemblies, devices, apparatus articles, compositions, methods, processes and techniques.

In this specification the word “comprising” is to be understood in its “open” sense that is the sense of “including” and thus not limited to its “closed” sense, that is the sense of “consisting only of. A corresponding meaning is to be attributed to the corresponding words “comprise, comprised and comprises” where they appear.

It will further be understood that any reference herein to known prior art does not, unless the contrary indication appears, constitute an admission that such prior art is commonly known by those skilled in the art to which the invention relates.

What is claimed is:

1. A non-invasive method for deriving cardiac output of a patient, the method comprising:
obtaining a first continuous waveform corresponding to an arterial pressure waveform at a first site on the patient using a first non-invasive sensor in contact with the patient;
measuring systolic and diastolic arterial pressures using a non-invasive pressure system in contact with the patient;
scaling and calibrating the first continuous waveform based on the measured systolic and diastolic arterial pressures;
determining mean arterial pressure and an arterial time constant from the scaled and calibrated continuous waveform;
deriving vascular compliance based on a pulse wave delay between two continuous waveforms corresponding to the arterial pressure waveform at different sites on the patient; and
calculating the cardiac output as a function of the mean arterial pressure, the vascular compliance, and the arterial time constant.

2. The non-invasive method of claim 1, wherein deriving vascular compliance comprises:
obtaining a second continuous waveform corresponding to the arterial pressure waveform at a second site on the patient using a second non-invasive sensor in contact with the patient; and
computing the pulse wave delay between the first and second continuous waveforms.

3. The non-invasive method of claim 1, wherein deriving the vascular compliance comprises:
obtaining a second continuous waveform corresponding to the arterial pressure waveform at a second site on the patient using a second non-invasive sensor in contact with the patient;
obtaining a third continuous waveform corresponding to the arterial pressure at a third site on the patient using a third non-invasive sensor in contact with the patient; and
computing the pulse wave delay between the second and third continuous waveforms.

4. The non-invasive method of claim 2, wherein the non-invasive sensors include at least one of an ultrasound Doppler sensor, an electrocardiogram, a microphone, or a combination thereof.

5. The non-invasive method of claim 3, wherein the non-invasive sensors include at least one of an infrared optical sensor, an ultrasound Doppler sensor, an electrocardiogram, a microphone, or a combination thereof.

6. The non-invasive method of claim 1, wherein the calculating of the cardiac output comprises:
multiplying the mean arterial pressure and the vascular compliance; and
dividing the multiplied mean arterial pressure and vascular compliance by the arterial time constant.

7. The non-invasive method of claim 1, wherein the arterial time constant is determined from an exponential fall in value of the scaled and calibrated continuous waveform after a dicrotic notch region.

8. The non-invasive method of claim 2, wherein the first site and/or the second site is at least one of a finger, a thumb, a toe, an earlobe, a forehead, or a site over an arterial vessel.

9. The non-invasive method of claim 3, wherein the second site and/or the third site is at least one of a finger, a thumb, a toe, an earlobe, a forehead, or a site over an arterial vessel.

10. The non-invasive method of claim 1 further comprising:
measuring the heart rate; and
calculating a stroke volume by dividing the calculated cardiac output by the measured heart rate.

11. The non-invasive method of claim 10, further comprising:
deriving cardiac contractility of the patient from a rate of change of value of an upswing of the scaled and calibrated continuous waveform,
deriving a rate of change of pressure from the cardiac contractility; and
deriving a rate of change in volume from the calculated stroke volume and the cardiac contractility.

12. The non-invasive method of claim 1, wherein the non-invasive pressure system is at least one of an electronic sphygmomanometer, or an inflatable cuff connected to a manometer with a pulse sound detector.

13. The non-invasive method of claim 12, wherein the pulse sound detector comprises at least one of a stethoscope or a microphone.

14. A system for deriving cardiac output of a patient, the system comprising:
a first non-invasive sensor for generating a first continuous waveform corresponding to the arterial pressure waveform at a first site on the patient;
a non-invasive means for generating at least one of a second continuous waveform corresponding to the arterial pressure waveform at a second site on the patient and a third continuous waveform corresponding to the arterial pressure waveform at a third site on the patient;
a non-invasive pressure system for measuring the arterial pressure of the patient;
a processing unit for:
scaling and calibrating the first continuous waveform based on the measured arterial pressure of the patient;
determining a mean arterial pressure and an arterial time constant from the scaled and calibrated continuous waveform;
deriving arterial compliance by determining a pulse wave delay between the first and second continuous waveforms or the second and third continuous waveforms; and
calculating the cardiac output as a function of the mean arterial pressure, the arterial compliance, and the arterial time constant; and
means for displaying at least the arterial pressure and the cardiac output.

15. The system of claim 14, wherein the non-invasive means include at least one of an infrared optical sensor, an ultrasound Doppler sensor, an electrocardiogram, a microphone, or a combination thereof.

16. The system of claim 14, wherein the non-invasive sensor includes at least one of an infrared sensor or an ultrasound Doppler sensor.

17. The system of claim 14, wherein the non-invasive pressure system includes at least one of an electronic sphygmomanometer, or an inflatable cuff connected to a manometer with a pulse sound detector.

18. An apparatus for deriving cardiac output of a patient, the apparatus comprising:
an input configured to:
receive a first continuous waveform corresponding to an arterial pressure waveform at a first site on the patient;
receive at least one of a second continuous waveform corresponding to the arterial pressure waveform at a second site on the patient and/or a third continuous waveform corresponding to the arterial pressure waveform at a third site on the patient;
obtain measured systolic and diastolic arterial pressure values; and
a processing unit configured to:
scale and calibrate the first continuous waveform signal based on the received systolic and diastolic arterial pressure values;
determine mean arterial pressure and time constant from the scaled and calibrated first continuous waveform;
derive arterial compliance based on a pulse wave delay between either the first and second continuous waveforms or the second and third continuous waveforms;
calculate cardiac output as a function of the mean arterial pressure, arterial time constant and vascular compliance; and
transmit the cardiac output and the mean arterial pressure to an output device.

19. The apparatus of claim 18 further comprising:
a memory device electrically coupled to the processing unit, the memory device configured to store at least one of the systolic and diastolic arterial pressure values, the vascular compliance, the arterial time constant, the mean arterial pressure or the cardiac output values.

20. The apparatus of claim 18, wherein the processing unit is further configured to:
compute cardiovascular parameters including at least one of stroke volume, heart rate, systemic vascular resistance, cardiac contractility, ECG, cardiac index, systolic pre-ejection period; and
transmit the computer cardiovascular parameters to the output unit.
